(19) United States
(12) Patent Application Publication
(10) Pub. No.: US 2025/0027209 A1
(43) Pub. Date: Jan. 23, 2025

(54) Biological Battery Reactor System in manufacturing Metal Oxide, generating electricity, and fixing CO2

(71) Applicant: Pei Zhang, Boston, MA (US)

(72) Inventor: Pei Zhang, Boston, MA (US)

(21) Appl. No.: 18/222,495

(22) Filed: Jul. 17, 2023

(51) Int. Cl. C25B 5/00; C25B 1/00; C25B 9/19; C25B 9/65; C25B 15/08

(52) U.S. Cl. CPC C25B 5/00; C25B 1/00; C25B 9/19; C25B 9/65; C25B 15/083

(57) ABSTRACT

Here we present a novel renewable industrial method to manufacture metal oxide compounds, such as ZnO. The process relies on active functional biological material including functional microorganisms and the relevant battery technology. Unlike traditional combustion manufacture process, which is heavily rely on intensive energy consumption and generates tremendous CO₂ during manufacture, this novel manufacture method generates renewable electricity instead. It reduces the energy consumption tremendously and reduces the operational cost. In the novel method, during manufacture, the biological catalysts promote electrochemical reactions in the biobattery reactor system. And with higher renewable power generation, more metal oxide is produced. And under the proper conditions with CO₂, light and certain conditions, the microorganisms can fix CO₂ and convert CO₂ to biofuel or multi-carbon compounds at the same time.

### FIELD OF INVENTION

This invention related to a novel renewable industrial metal oxide producing method, which simultaneously generates electricity, fixes CO₂, and forms multi-carbon compounds. This invention relates to metal oxide production; biological battery technology; advanced manufacture method; renewable technology; clean tech; nano technology of forming nano structure during metal oxide production; CO₂ fixation; decarbonation; low carbon footprint

### BACKGROUND

### Metal Oxides

Metal oxides are one of the most important and widely characterized solid catalysts. Considered as heterogeneous catalysts and are applied for acid-base and redox reactions. They are applied widely in various reactions (Scheinost 2005). Metal oxide-based photocatalysts have been used for photocatalytic degradation of pharmaceuticals and personal care products. Also, metal oxides have an important role in organic transformations, due to their simplicity in handling, decreased reactor and plant corrosion problems, cost effectiveness and because most of the metal oxides are reusable and recyclable. ZnO is a white inorganic compound that is used widely in pharmaceuticals, rubber, ceramics cosmetics, chemicals, and glass industries. The growth of these end-use industries is expected to fuel the global zinc oxide market demand over the forecast period.

### Market of Metal Oxide

The global metal oxide nanoparticles market is expected to witness strong growth over the forecast period owing to increasing demand from major end-use industries, including adhesives & sealants, automotive, energy, electronic & optoelectronics, aerospace, and packaging. Strong growth of these end-use industries anticipated to trigger the metal oxide nanoparticles market growth over the forecast period. Owing to properties of metal oxide nanoparticles such as electronic, mechanical, optical, and chemical catalytic properties they find vast applications in high-tech industries. Based on various metal oxides used, Zinc oxide nanoparticles market has been witnessing strong growth owing to its increasing application in sunscreen lotions and cosmetics on account of its high UV protection properties. in addition, zinc oxide nanoparticles are used in textiles, electronics, paints, and coatings owing to its anti-bacterial, anti-fungal, anti-corrosion, and catalytic properties. ZnO is also used for the vulcanization of rubber to increase the durability of rubber. The tire industry further uses this vulcanized rubber for the manufacturing of tires.

Magnesium oxide nanoparticles market is expected to witness strong growth over the next seven years on account of increasing demand from furnace linings, construction and ceramics, pharmaceuticals, and food additives. Moreover, the titanium oxide nanoparticles market is anticipated to witness significant growth in light of its rising applications in medical & pharmaceuticals and cosmetics sectors. North America was the largest market for metal oxide nanoparticles market owing to high venture capital investments in R&D activities to enhance nanoparticles product applications. Huge demand from end-use industries including automotive and pharmaceutical is likely to boost its market in the region. In 2018, the rubber industry consumed more than half of the ZnO produced around the world due to the growing production of tires. Rising demand for rubber in the tire, as well as non-tire applications, is expected to drive the demand for zinc oxide consequently. Amid the COVID-19 crisis, the global market for Nano-Metal Oxides estimated at US$5.3 Billion in the year 2020, is projected to reach a revised size of US$9.3 Billion by 2027, growing at a CAGR of 8.3% over the analysis period 2020-2027. The global zinc oxide (ZnO) market size is projected to grow from USD 4.4 billion in 2019 to USD 5.7 billion by 2024, at a compound annual growth rate (CAGR) of 5.4%, during the forecast period.

### Manufacture of Metal Oxide (Use ZnO as an Example)

In the market of metal oxide, manufacture of each material has different manufacture. Zinc oxide has very strong growth in the market due to its increasing demand in the market. For industrial use, ZnO is produced at levels of 10⁵ tons per year by three main processes:

First is through indirect process. By weight, most of the world's zinc oxide is manufactured via French process. Metallic zinc is melted in a graphite crucible and vaporized at temperatures above 907° C. (typically around 1000° C.). Zinc vapor reacts with the oxygen in the air to give ZnO, accompanied by a drop in its temperature and bright luminescence. Zinc oxide particles are transported into a cooling duct and collected in a bag house. Its product normally consists of agglomerated zinc oxide particles with an average size of 0.1 to a few micrometers.

Second is through direct process. The direct or American process starts with diverse contaminated zinc composites, such as zinc ores or smelter by-products. By heating with a source of carbon such as anthracite to produce zinc vapor, which is then oxidized as in the indirect process. Because of the lower purity of the source material, the final product is also of lower quality in the direct process as compared to the indirect one, the French process.

Third is through wet chemical process, a small amount of industrial production involves wet chemical processes, which start with aqueous solutions of zinc salts, from which zinc carbonate or zinc hydroxide is precipitated (time consuming). The solid precipitate is then calcined at temperatures around 800° C. Need to heat up to high temperature to transform Zn(OH)₂ to ZnO, which increased an extra step and operation cost. The hydrothermal method is one of the simplest and most available methods suitable for synthesis of ZnO nanorods on polymeric substrates (Semenova 2017). Usually, NaOH, KOH, ammonium hydroxide NH₄OH or hexamethylenetetramine C₆H₁₂N₄ are used as an alkali. During the reaction, a zinc precursor is hydrolyzed, and, as a result, Zn²⁺ ions appear and react with the anions of alkali OH⁻. During the reaction, various hydroxides can be formed: ZnOH⁺ (aqueous), Zn(OH)₂ (aqueous), Zn(OH)₂, Zn(OH)₃⁻ (aqueous) and [Zn(OH)₄]²⁻. The growth of zinc oxide micro- and nanostructures occurs in the direction of c-axis, resulting in formation of an anisotropic structure. And Polyethylene terephthalate (PET) with ITO (60 Ohm) layer (Sigma Aldrich) was used to form ZnO nanostructures on flexible substrates (Semenova 2017).

The process is normally initiated by the formation of Zn(OH)₂ followed by their transformation into ZnO. Based on the SEM observations, mechanisms of the phase transformation from Zn(OH)₂ to ZnO have been revealed as three types, namely dissolution-reprecipitation, in situ crystallization and solid-solid phase transformation. The loss of water from the lattice during the solid-solid phase transformation is suggested. Zn(OH)₂ readily dissolves at higher temperature and a hole could be dug where defects are rich. Dehydration of Zn(OH)₂ at the bottom of the hole leads to the formation of ZnO nuclei. Growth units of Zn(OH)₄²⁻ provided by dissolving Zn(OH)₂ can reach the nucleus and facilitate the further growth of ZnO needles. Once the growth of ZnO is initiated, the remaining parent Zn(OH)₂ serves simply as a template holding the needle crystals of ZnO until its complete dissolution (Wang 2011).

Wet chemical process is expected to be the fastest-growing process for ZnO between 2019 and 2024. The wet chemical process is projected to grow at the fastest rate during the forecast period. ZnO Grade produced by this process are very fine in size has good dispersion and slow settling rate. Due to the more effective activation offered by the product, it is called “Active ZnO”, which has superior quality than the ones produced by the French process. ZnO occurs in the mineral zincite, but most of the commercial product is made by the high-temperature oxidation of metallic zinc or zinc ores, which produces low quality ZnO and has high cost on energy, especially nowadays when the price of energy becomes higher and higher.

### Key Market Players

The key players in the zinc oxide market are US Zinc (US), Zochem (US), EverZinc (Belgium), ZM Silesia (Poland), Akrochem (South Africa), Rubamin (India), Pan-Continental Chemical (Taiwan), J.G. Chemicals (India), Upper India (India), and Suraj Udyog (India). These players have strong R&D and focus on producing high-performance products to meet the demands of end-users. US Zinc is one of the leading producers of ZnO in the world. It manufactures almost 40 different grades of ZnO, which caters to various applications such as rubber, ceramics, chemicals, and animal feed. In recent years, the developed economies such as Europe has brought in regulation which limits the use of ZnO in application like animal feed. These regulations will create an opportunity for the company in developing economies like the Middle East & Africa, which can be tapped easily due to the global reach of the company.

### Energy and Global Warming Issues

The industrial production and human civilian activities demand tremendous energy consumption. Traditional energy generation processes, such as burning coal, burning fossil fuels like oil, natural gas and petroleum generate lots of air pollutions causes global warming. With the rise in industrial pollution, global warming has been increasing at a steady pace. Smoke and greenhouse gases are being released by industries into the air, which causes an increase in global warming including abnormal weather changes, melting of glaciers, extinction of polar bears, floods, tsunamis, hurricanes etc.

Technologies related to renewable energy, such as solar, wind and tie, are promising. They reduce the reliance of the traditional energy generation process, which involves combustion of natural resources and generation of global warming gas. However, many industrial production processes still involve steps of burning natural resources to generate high heat and high temperature. Design and invention of renewable industrial manufacture processes to avoid continuous generation of tremendous global warming gas are in urgent demand.

### Biological Battery (Biobattery) Technology

Biological batteries utilize biological material to catalyze cathode electrodes performance in metal air battery to remove the reliance of the battery functions on harsh and toxic chemicals (Zhang 2020). The biological battery (biobattery) can be used to manufacture interesting metal oxide material, such as ZnO, MgO, Al₂O₃, etc and generating electricity at the same time. The functional microorganisms with biological materials have interesting metabolic pathways on photo-mixotrophic capabilities for biosynthesis. Wherein they can fix CO₂ for biosynthesis (Wan 2015).

### Potential Pathways of the Functional Microorganisms

The Calvin cycle is part of photosynthesis, which occurs in two stages. In the first stage, chemical reactions use energy from light to produce ATP and NADPH. In the second stage (Calvin cycle or dark reactions), carbon dioxide and water are converted into organic molecules, such as glucose. Although the Calvin cycle may be called the “dark reactions,” these reactions don't occur in the dark or during nighttime. The reactions require reduced NADP, which comes from a light-dependent reaction. The Calvin cycle consists of: Carbon fixation—Carbon dioxide (CO₂) is reacted to produce glyceraldehyde 3-phosphate (G3P). The enzyme RuBisCO catalyzes the carboxylation of a 5-carbon compound to make a 6-carbon compound that splits in half to form two 3-phosphoglycerate (3-PGA) molecules. The enzyme phosphoglycerate kinase catalyzes phosphorylation of 3-PGA to form 1,3-biphosphoglycerate (1,3BPGA). Reduction reactions—The enzyme glyceraldehyde 3-phosphate dehydrogenase catalyzes reduction of 1,3BPGA by NADPH. Ribulose 1,5-bisphosphate (RuBP) regeneration—At the end of the regeneration, the net gain of the set of reactions is one G3P molecule per 3 carbon dioxide molecules.

Glycolysis is the metabolic pathway that converts glucose C₆H₁₂O₆, into pyruvic acid, CH₃COCOOH. The free energy released in this process is used to form the high-energy molecules adenosine triphosphate (ATP) and reduced nicotinamide adenine dinucleotide (NADH). Glycolysis is a sequence of ten reactions catalyzed by enzymes. It is a metabolic pathway that does not require oxygen. The wide occurrence of glycolysis in other species indicates that it is an ancient metabolic pathway. Indeed, the reactions that make up glycolysis and its parallel pathway, the pentose phosphate pathway, occur in the oxygen-free conditions of the Archean oceans, also in the absence of enzymes, catalyzed by metal. The pentose phosphate pathway (also called the phosphorgluconate pathway and the hexose monophosphate shunt and the HMP Shunt) is a metabolic pathway parallel to glycolysis. It generates NADPH and pentoses (5-carbon sugars) as well as ribose 5-phosphate, a precursor for the synthesis of nucleotides. While the pentose phosphate pathway does involve oxidation of glucose, its primary role is anabolic rather than catabolic. The pathway is especially important in red blood cells (erythrocytes).

TCA cycle (tricarboxylic acid cycle) or the Krebs cycle—is a series of chemical reactions to release stored energy through the oxidation of acetyl-CoA derived from carbohydrates, fats, and proteins. The TCA cycle is used by organisms that respire (as opposed to organisms that ferment) to generate energy, either by anaerobic respiration or aerobic respiration.

### Synthesis of Nano Metal Oxide

Controlled precipitation is a widely used method of obtaining zinc oxide, since it makes it possible to obtain a product with repeatable properties. The method involves fast and spontaneous reduction of a solution of zinc salt using a reducing agent, to limit the growth of particles with specified dimensions, followed by precipitation of a precursor of ZnO from the solution. At the next stage this precursor undergoes thermal treatment, followed by milling to remove impurities. The process of precipitation is controlled by parameters such as pH, temperature, and time of precipitation. This method has a fairly high temperature in calcination at 450° C. for 3 h. The ZnO obtained had a narrow range of particle sizes, from 9 to 20 nm. Prepare ZnO nanoparticles at high reaction temperatures. Some of these synthesis processes usually require long growth time, high temperature and poor quality control. To circum-vent these challenges, the chemical bath deposition (CBD) method was used to prepare ZnO at a low growth time and temperature (Fokotsa 2015). CBD is easy, cheap and user friendly since parameters such as pH, time, concentration and temperature can easily be varied Chemical bath deposition (CBD) is the name given to a variety of techniques that produce films of solid inorganic, non-metallic compounds on substrates by immersing the substrate (once, or repeatedly) in a precursor solution (often aqueous). The transformation of Zn(OH)₂ to the well-known hexagonal wurtzite structure of ZnO is with the increase in growth temperature to provide a simple and efficient approach for the synthesizing of the ZnO with flakes-like morphology. In the detail of a one method, four sets of samples were prepared under different growth temperature (55, 65, 75 and 80° C.). The resulting mixture was continuously stirred until the white precipitates were formed. The reaction was allowed to run for 10 min for all growth temperatures. It was later allowed to stabilize and cool down overnight. Finally, the precipitate was collected, washed several times with ethanol and acetone to remove the residue and desiccated for a maximum of three days to ensure that powders were dried prior to characterization (Fokotsa 2015). The ZnO nano-powders were prepared successfully by CBD method at various growth temperatures. The increase in the growth temperature improved the thermal stability of ZnO nanophosphors. The melting temperatures were observed to decrease due to crystallization of ZnO. From the XRD Zn(OH)₂ decomposed into ZnO and crystallinity was improved. Morphological changes were observed where spherical and flakes-like shapes accumulated to form the complete flakes-like. The results showed that the band gap energy of the ZnO can be tuned by varying the growth temperature (Fokotsa 2015).

In processes of synthesis of nano powders based on precipitation, it is increasingly common for surfactants to be used to control the growth of particles. The process was carried out at room temperature, and the resulting powder was calcined at 500° C. to remove residues of the surfactant. The product was highly crystalline ZnO with a wurtzite structure and with small, well-dispersed spherical nanoparticles in size of 50 nm.

The hydrothermal method does not require the use of organic solvents or additional processing of the product (grinding and calcination), which makes it a simple and environmentally friendly technique. In hydrothermal synthesis processes, one hydrothermal method is to synthesize nanocrystalline zinc oxide with different particle shapes and sizes. A reaction of zinc acetate or nitrate with a suitable hydroxide (LiOH, KOH, NH₄OH) produced the precursor Zn(OH)₂·nH₂O. Using an autoclave, in isothermal conditions or at variable temperature (120-250° C.). Dehydration of the precursor, followed by recrystallization, produced crystallites of ZnO with a hexagonal structure and sizes of 100 nm-20 μm.

Another method is using suspension obtained from a solution of Zn (CH₃COO)₂·2H₂O and neutralized using different quantities of a solution of NH₄OH underwent hydrothermal treatment in an autoclave at a temperature of 160° C. It was found that the pH affected the size and shape of the ZnO particles. Maturing of the original aqueous suspension for 7 months (at a pH of 10, and at room temperature) led to the appearance of aggregates consisting of ZnO particles with sizes between ˜20 and ˜60 nm (Radzimska 2014).

### SUMMARY

Metal oxide is a group of very important industry products that plays important role everywhere in our life. And the amount needed is growing over the time. The major industrial manufacture process is still in the high energy intensive and high CO₂ emission mode. As the energy resources from nature be consumed over the time, the energy becomes more and more expensive, while the cost of environment pays off with worse global warming and many other immerging global environmental issues, which threatened the life of human being on earth in return. It is urgent and important to start advanced manufacture with higher efficiency, lower energy consumption and lower the negative environmental impact. And our nature has been teaching us in our history that the best way to solve this critical issue is to borrow the method from the nature.

Here we present a novel renewable microbial metal oxide industrial production method, using Biological Battery Reactor System to manufacture valuable metal oxide compounds, such as ZnO, MgO, Al₂O₃ etc. The process relies on active functional biological material and relevant battery technology. Comparing to the traditional combustion manufacture method, this novel industrial production method reduces energy consumption, and generates electricity in the process. It reduces the operational cost. This method may use biological catalyst compounds to convert CO₂ to useful compounds at the same time. Biological battery technology has been developed in recent years. The technology presents the novelty in applying the functional microorganisms into modern industrial manufacture process and makes the process renewable with value adding proposition. The biological battery technology can be directly applied in industrial manufacturing process to overcome the challenges that the conventional industry is facing, and to start a time of renewable advanced manufacture.

Biological components play key role in overcoming the challenges from industrial needs and environmental issue. Two billion years on earth, microorganisms utilize CO₂, light and water to generate oxygen and life. And the oxygen in the atmosphere was largely rely on their photosynthetic activities. Nowadays, we will utilize microorganisms again to convert tremendous surplus industrial byproduct CO₂ to useful multi-carbon carbons, oxygen, and biomass. Biological battery technology has been developed over the past 10 years. The technology presents the novelty in embedding the functional microorganisms to syntrophic growth with the modern battery technology and making it renewable with variety of value adding functions and potentials. The functional microorganisms in biobattery reactor have been investigated and enhanced performance 10 times in the past awarded research from NSF (Zhang 2012). After 7 years research, the microorganisms have been successfully applied into biobattery system as cathode catalyzer. The biological battery technology can be efficiently applied into metal oxide industrial manufacturing process to overcome the challenges and start a new age of renewable advanced manufacture of metal oxide.

There are several key points of the invention need to address. Design of the system such as FIGS. 6 and 7 for the biobattery reactor system to be stand-alone operational system as much as possible without too much relay on external supply of labor and energy. With the easy control influence tank and separation of the electrolyte and the biological medium in the system to simplify the needs of post product treatment with higher efficiency. The design of the recycle part of the system, makes a full use of functional biological medium and maximize the output amount. Also, the effluent electrolyte is also recycled after obtaining the precipitation material. The entire system is highly efficiency and almost nothing left to throw away or to be disposed. Also, with value adding propositions of electricity generation and CO₂ fixing, the manufacture process opens the potential of higher value generation at the same time, which compensate the intrinsic shortcoming of slow and steady of the biological production rate under the neutral conditions.

One challenge of battery technology and many other electrochemical technologies is the electron collection and connection. Lots of energy was wasted during electron transport process, especially during the scaling up process for industrial scale manufacture. In this invention, an electron collection and transport assistant design of cheese cage electricity collection structure (CCEC) is presented as shown in FIG. 3,4,5.

One critical technical point is the formation of nano structure of the metal oxide product. There has been research developed in monitoring the formation of nano structure during metal oxide product generation. Nano structure is important for the function of metal oxide. Nowadays developing nano structure efficiently during metal oxide production is still a challenge. This invention presents a novel technology to form nano structure efficiently during metal oxide production. And the technology is scaling to large scale manufacture. Using battery and biobattery technology to develop nano structure during metal oxide production is a novel method. The process is also cost effective in shortening the time and the cost of forming metal oxide with nano structures. Many traditional methods involve high temperature and adding extra agents or chemicals in production, which will cause extra wash and cleaning after the production. But biobattery technology can obtain the metal oxide without high temperature nor adding extra chemicals and the nano structure is generated during the process.

### BRIEF DESCRIPTION OF THE DRAWINGS

The objects and features of the invention can be better understood with reference to the drawings described below, and the claims.

FIG. 1a (is) Illustrates a cross section view of all the components on one biological battery reactor design in producing metal oxide material, electricity, and fixing CO₂ according to an embodiment of the present invention.

FIG. 1b (is) Illustrates a side view of the components of a biological battery reactor in producing metal oxide material, electricity, and fixing CO₂ according to an embodiment of the present invention.

FIG. 2 (is) Illustrates a side view of all the components on one biological battery design in producing metal oxide material, electricity, and fixing CO₂ according to an embodiment of the present invention.

FIG. 3 (is) Illustrates the cheese cage anode electricity collection structure according to an embodiment of the present invention.

FIG. 4 (is) Illustrates the cheese cage cathode electricity collection structure according to an embodiment of the present invention.

FIG. 5 (is) Illustrates a schematic top view of a biological battery metal oxide material manufacture design with an anode cheese cage electricity collection structure and a cathode cheese cage electricity collection structure installed.

FIG. 6 (is) Illustrates a schematic view of a biological battery metal oxide material manufacture design: Inside the cheese cage electricity collection, including biological catalyzer charging and recycle path, electrolyte charging path, CO₂ charging path, as well as effluent biofuel collection.

FIG. 7 (is) Illustrates a schematic view of a biological battery producing metal oxide material manufacture design: Outside cheese cage electricity collection structure, including biological medium charging and recycle path, electrolyte charging path, CO₂

FIG. 8 (is) Illustrates a schematic view of a treatment of the effluent of the electrolyte of a biological battery reactor to produce metal oxide as one product.

### DETAILED DESCRIPTION

### The Functional Microorganisms

The invention can be described specifically and illustrated by presenting one or more preferred embodiments in the paragraphs that follow. At least one preferred embodiment of the present invention provides for incorporating into an electrode one or more strains of microorganisms that can enact cathode catalyzing performance. The microorganisms catalyze the oxygen redox reaction on cathode forming hydroxide group and react with cations from anode forming metal hydroxide precipitation and through low heat dry to form metal oxide products. In at least one embodiment, the process can be used to form metal oxide material, such as ZnO, MgO, Al₂O₃ etc. Unlike the traditional manufacture processes, which are energy intensive and generates lots of CO₂, especially combustion method, the biobattery metal oxide manufacture process can generates electricity, fix CO₂ while producing useful end byproducts clean. Rather than consume electricity and generating CO₂ like during the traditional manufacture process.

In at least one embodiment, the microorganisms could be photosynthetic mixotrophic microorganisms. They can fix CO₂ and convert carbon dioxide into biofuel or useful multi-carbon compounds under light and aerobic conditions (Wan 2015).

In at least one other embodiment, a microorganism can utilize the electrons from the cathode electrode as energy source and carbon from environment as carbon source to catalyze the electrochemical reactions near or on the cathode and promote the energy generation from the battery. The microorganisms also catalyze the electrochemical reaction in the battery in producing metal oxide compounds. (Zhang 2020)

In at least one other embodiment, a microorganism can utilize the electrons from the cathode electrode as well as the energy from photons as energy source and carbon from environment as carbon source to catalyze the electrochemical reactions in battery therefore promote the energy generation as well as the metal oxide productions in the system. (Zhang 2020) The microorganisms can be utilized in clean industrial metal oxide production while fulfil decarbonate and electricity generation at the same time.

In at least one embodiment, the reaction medium can be configured to contain carbon dioxide as a source of carbon and to be oxidized; a source of electrical energy, a source of light, the source of electrical energy connected to the cathode contact terminal; and the biological medium adjacent to at least one surface of the cathode electrode by way of the working medium.

Depending on the specific microorganism(s) utilized, variables to be considered in forming products include, for example, among others, without limitation: nutrient constituents and concentrations, medium, temperature, gas flow rate, liquid flow rate, access of light, access of CO₂, access of air, reaction pH, agitation rate (if utilizing a Continuously Stirred Tank Reactor), hydraulic retention time, inoculums concentration, maximum substrate (introduced gas) concentrations to avoid inhibition, and maximum product concentrations to avoid inhibition.

The invention also provides, in an alternative aspect, one or more pure cultures of the microorganisms can be describable.

### Biological Battery (Biobattery) Reactor Design for Manufacturing Metal Oxide

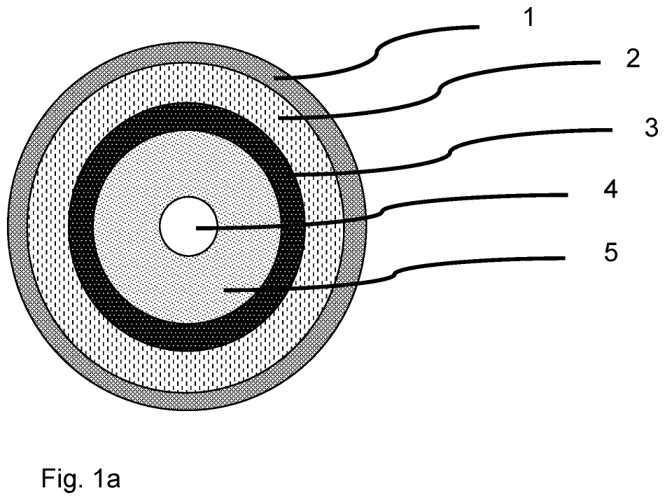

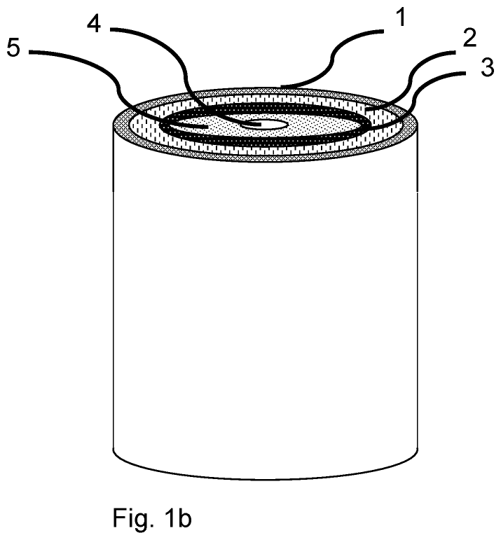

To promote production rate of metal oxide product and power output, it is important to have a proper biobattery design to provide better conditions for the functional microorganisms to grow and catalyze the electrochemical reactions. In one embodiment, wherein a reaction vessel, an anode electrode and a cathode electrode are all in cylinder shape as shown in FIGS. 1a and 1b. Wherein the anode electrode 1 is the outside cylinder; and the cathode electrode 3 is the inside cylinder, wherein inside cathode circle. encloses a biological medium 5, wherein in the middle of the biological medium is a light source 4 in the case of the biological medium contains photosynthetic CO₂ fixing microorganisms. these microorganisms need photons to generate biological energy for fixing CO₂ providing biochemical reactions and biomass growth. Certain microorganisms can use the electrons from electrode as energy source. The biobattery reactor contains an electrolyte 2. In one embodiment, the electrolyte can mix with the biological medium. In another embodiment the electrolyte can separate from the biological medium. The electrolyte is in between the anode and cathode cylinders and contacting the anode and cathode electrodes directly. The biological medium is inside the cathode cylinder if separate from the electrolyte. A supporting and secure structure is at outside the anode electrode cylinder to physically support the shape of the reactor.

The advantages of this design are firstly for photosynthetic microorganisms, it is easier to control and monitor the light source, keep it constant for a better and consistent CO₂ fixation as well as manufacture of metal oxide and energy generation. Secondly this design separates the microorganism from the electrolyte and metal oxide product, which makes it better to control the electrolyte mass flow and the biological medium mass flow, recycle and keep a long-term better performance for both and it makes it easier to clean and wash the final metal oxide product for a better quality. The disadvantage of this design is firstly, it limits the surface area of cathode and potentially limits the power output and energy density of the system. Secondly, the use of anode metal might not be as efficient and cost effective due to it may need to customize the material.

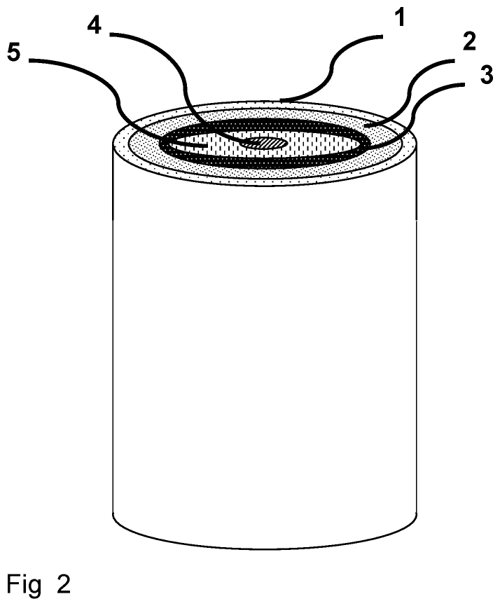

In one other embodiment, wherein a reaction vessel as shown in FIG. 2, an anode and a cathode electrode are all in cylinder shape. The anode electrode 4 is the inside cylinder. The cathode electrode 3 is the outside cylinder. The outside cylinder 1 encloses the cathode electrode from outside with the biological medium 2 surrounding the cathode electrode and within the outside cylinder. The outside cylinder can be transparent in favored of photosynthetic CO₂ fixing microorganisms. The electrolyte 5 can mix with biological medium in one embodiment and separate from the biological medium in one other embodiment. The electrolyte is in between the anode and cathode and contacting the anode and cathode electrodes directly.

The advantage of this design is firstly it provides larger surface area for cathode have better power output and energy density. Secondly, it uses the anode material more efficiently and cost effectively. The disadvantage is firstly, it is not easy to control and monitor the light source, therefore may impact the performance of the functional photosynthetic microorganisms. Secondly the anode in the center be reacted and may lost the connection at certain point cause the loss of the performance in a sudden.

### Biological Battery (Biobattery) Reactor System for Manufacturing Metal Oxide

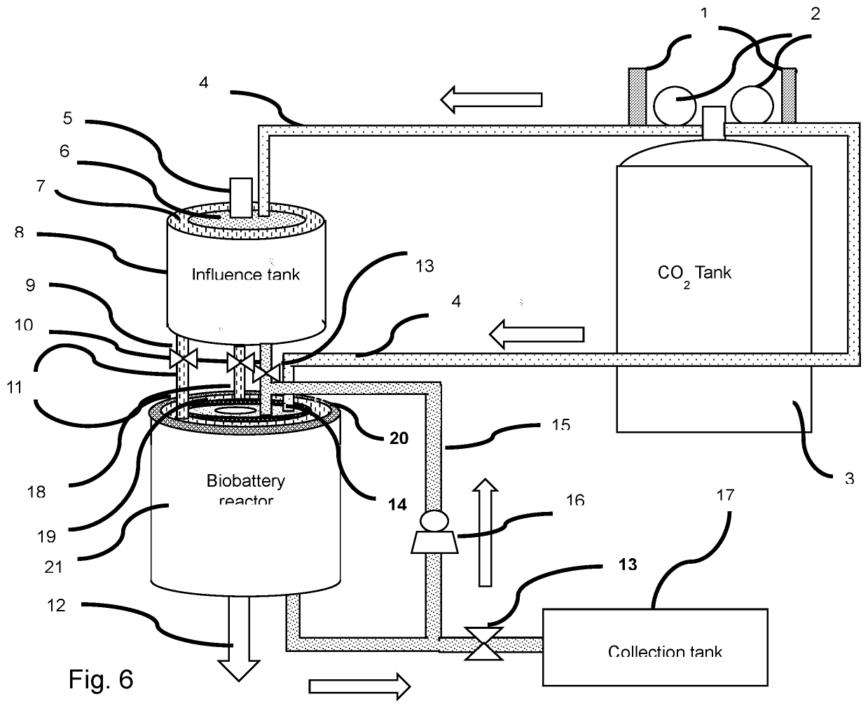

A biological battery reactor system in manufacturing metal oxide product is a system has mixed function of metal oxide production, energy generation and potentially CO₂ fixation. In one embodiment, the overall system combing the electrochemical battery technology and microbial syntrophic growth technology, displayed in an embodiment of biobattery system design as shown in FIG. 6. Where the functional microorganisms are grow in a reaction vessel with an anode electrode 20 and a cathode electrode 14 disposed therein, the anode electrode having at least one surface and an anode electrical contact terminal, the cathode electrode having at least one surface and a cathode electrical contact terminal, the cathode electrode having a biological medium 19, which contains biologically active material, adjacent to at least one modified surface of the cathode electrode and in electrical communication therewith, the reaction vessel configured to contain a working medium, electrolyte 17, for anode and cathode having mobile ions and in contact with the cathode electrode and the anode electrode. the biological medium 19 configured to contain dissolved carbon dioxide as a source of carbon for cathode with CO₂ purging through pipeline 4. to near the cathode side, such that the gas mixes dissolve into the medium, and maintaining pH around neutral and maintaining other conditions appropriate for permitting the bacterium to catalyze the oxidation reaction with oxygen as electron acceptor and CO₂ as the carbon source. Also, there is a continuous access or supply of air or CO₂ into the biological medium in biological medium chamber 6 in influence tank as in FIG. 6.

### Medium Flow

A batch or a flow of medium into the reactor from an influence tank 8. The electrolyte from electrolyte chamber 7 in influence tank flow into electrolyte chamber 18 in biobattery reactor and flow out of the reactor through line 12. The biological medium in influence tank biological medium chamber 6 flow into the biological medium chamber 19 in biobattery reactor through pipeline 10 and flow out of the reactor through a pipeline 15 to recycle or move to collection tank 17.

In one embodiment, the working medium and the biological medium is one solution. It contains electrolyte, mobile ions, and cathode electrode active biological material (U.S. Utility patent Ser. No. 10/658,692, Awarded May 2020) In this embodiment the influence working medium flow into the biobattery reactor, wherein electrochemical reactions happened. And after the metal oxide compounds formed in certain volume, the working medium including the electrolyte, the metal oxide precipitations and biological medium will be discharged as effluent to a wash and clean tank, wherein the metal oxide precipitation will be washed to remove other chemicals and particles. Next step is to go through sieve, wherein the large metal oxide precipitations will be retained, and the small compounds and particles such as microorganisms will be sifted out. After filtration, the product will be air dry or dry through low heat to remove water molecules and the final product will be packed.

The wash out solution from the above process can be recycled after recovery via filtration through 0.2 μm filter adding ions and adjust pH then use as electrolyte in the biobattery or properly dispose.

In one other embodiment, the working medium electrolyte and the biological medium are separate solutions and are enclosed in separate compartment in the biobattery system. In FIGS. 1a and 1b, the electrolyte 2 are separate from biological medium 5.

In FIG. 6, a batch or a flow of medium from an influence tank 8 into the biobattery reactor 21. The electrolyte from electrolyte chamber 7 in influence tank flow into electrolyte chamber 18 in biobattery reactor through pipeline 11 and flow out of the reactor through line 12. The biological medium in influence tank biological medium chamber 6 flow into the biological medium chamber 19 in biobattery reactor through pipeline 10 and flow out of the reactor through a pipeline 15 to recycle or move to collection tank 17.

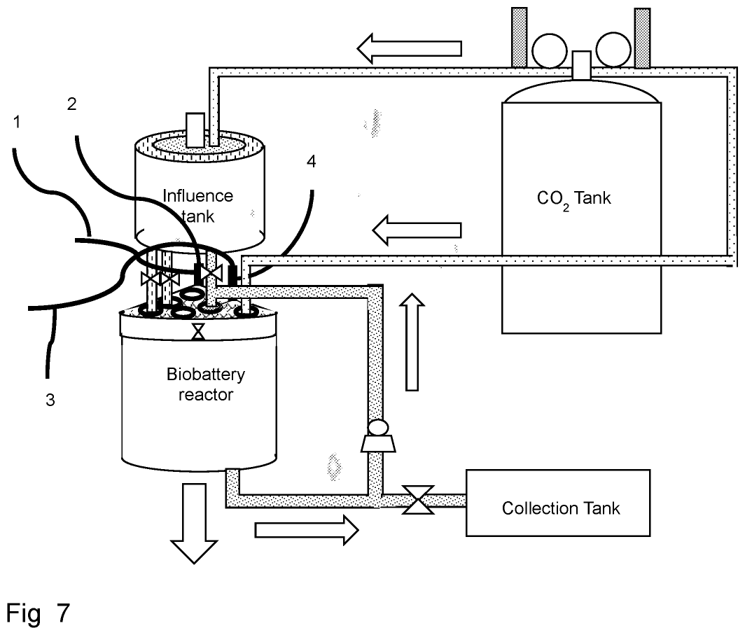

The biological medium system can be a flow through system, as in FIGS. 6 and 7, in which a medium with biological active material flow from the biological medium compartment 6 in the influence tank through connecting pipeline 10 with a control valve 13, and into the biobattery reaction vessel 21, wherein biological medium catalyzing electrochemical reactions to form metal oxide, electricity and for certain microorganisms, biofuel or multi-carbon compounds as well. The effluent biological medium flow into a biological medium recycling pipelines 15, wherein the pipeline is in transparent or semi-transparent material to allow certain CO₂ fixing microorganisms to grow and keep alive during transportation. The effluent biological medium can be recycled back to the biological medium enclosed in cathode electrode in biobattery reactor or discharged to a collection tank 17 through a control valve 13 to extract useful compounds. The biobattery reactor can be single stand or stack up or parallel with multiple reactors sharing the same influence tank and effluent collection tank. The medium can be in the form of liquid or gel.

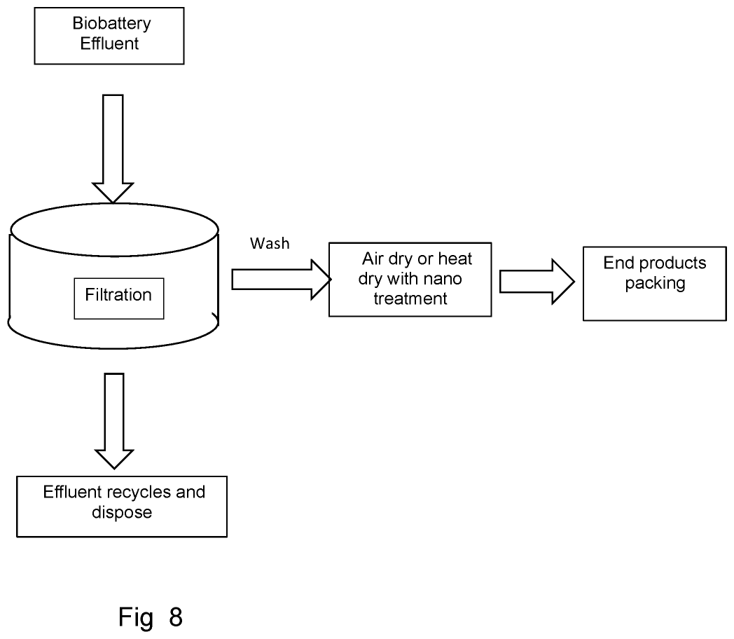

In regarding to the flow of the electrolyte. In the embodiment that the electrolyte is separated from the biological medium, as shown in FIGS. 1a and 1b, the electrolyte 7 in the influence tank 8 is separated from the biological medium 6. The electrolyte flow into the electrolyte compartment 18 in the biobattery reactor 21 through pipeline 11, and controlled by valve 13. In the biobattery reactor, the electrolyte is a solution in contact with the anode and cathode directly. It contains chemical compounds, mostly are ions and have major electrochemical reactions happened in it. After the metal is oxidized on anode and the oxygen is reduced on cathode, anions and cations migrate inside the electrolyte towards each other and form metal hydroxide precipitations mostly under the controlled conditions, such as pH, temperature, compounds in the electrolyte. At the same time, electrons are moved from cathode to anode via an external circuit through resistances or stored in another energy storage devices. The anode will be reduced over the time after the electrochemical reaction and the precipitation will be accumulated in the biobattery over the time. When certain volume of metal hydroxide precipitation formed, the control valve 13 will be opened. The precipitation, and the electrolyte will move out of the biobattery reactor into a wash and clean tank as shown in FIG. 8, wherein the precipitation will be washed and cleaned to remove other chemicals and particles. Next step is the filtration, wherein the large precipitations will be retained, and the small compounds and particles will be filtered through. After filtration, the precipitation will be washed, air dry or dry through low heat to remove water molecules. The product should have nano structure formed. But in case if the nano structure is not satisfied, nano treatment will be performed to generate better nano structures on the final metal oxide product. Then the final product will be packed.

The wash out solution from the above process can be recycled after recovery via filtration through 0.2 μm filter adding ions and adjust pH then use as electrolyte in the biobattery or properly dispose.

In one embodiment, Zn(OH)₂ readily dissolves at higher temperature, for example at 80° C. and a hole could be dug where defects are rich. Dehydration of Zn(OH)₂ at the bottom of the hole leads to the formation of ZnO nuclei. Growth units of Zn(OH)₄ provided by dissolving Zn(OH)₂ can reach the nucleus and facilitate the further growth of ZnO needles. Once the growth of ZnO is initiated, the remaining parent Zn(OH)₂ serves as a template holding the needle crystals of ZnO until its complete dissolution. mechanisms of the phase transformation from Zn(OH)₂ to ZnO have been revealed as three types, dissolution-reprecipitation, in situ crystallization and solid-solid phase transformation. The dehydration during the solid-solid phase transformation is supposed to take place between every two nearest hydroxyl groups in the Zn(OH)₂ lattice.

### CO₂ Fixation

The present invention provides, in at least one embodiment, a process for producing useful multi-carbon compounds, comprising biofuel: providing a continuous flow of air and/or a gas comprising carbon dioxide into a bioreactor, said bioreactor containing anode and cathode electrode, an aqueous nutrient biological medium and the microorganisms on the cathode side; directing a flow of the aqueous biological medium into the cathode side; and converting the gas in the medium by the microorganism at a pH around neutral and under conditions that permit converting the CO₂ to biofuel and useful multi-carbon compounds. Referring to one embodiment of a biological battery producing metal oxide material manufacture design of FIG. 6 and FIG. 7, CO₂ in pipe 4 is continuously purged into the biological medium 6 in the influence tank 8 as well as into the biological medium 19 of the biobattery reactor. The CO₂ gas is flow from the CO₂ tank 3 and delivered through pipe 4. The gas flow was controlled by regulator 2 correspondingly and through bubble counter 1 to monitor and deliver the CO₂ gas to the influence tank 8 and biobattery reactor 12 consistently and precisely. CO₂ flow rate is crucial for the biomass growth, therefore impact the production of the metal oxide compounds, the electricity and biofuel or multi-carbon compounds. This is because insufficient CO₂ supply may cause the inhibition of growth of the microorganisms during photosynthetic mixotrophic growth without sufficient carbon source. But over supply of CO₂ may cause pH change of the biological medium as well as the physiological growth condition of the functional group.

In one preferred embodiment the biofuel or multi-carbon compounds can be harvested from discharge the aqueous biological medium from the biobattery reactor. Referring to one embodiment of a biological battery producing metal oxide material manufacture design of FIG. 6 and FIG. 7, the effluent of the biological medium from the biological medium tank 19 in the biobattery reactor is recycled back to the biological medium 6 in the influence tank 8, through pipeline 15. A circulator pump 16 is installed on pipeline 15 to assist the circulation. The biofuel or multi-carbon products as well as biomass can also be discharged to the biofuel collection tank 17 through a control valve 13. In one preferred embodiment, the pipelines 15 and 10 are transparent. Influence biological medium can be directly added into influence tank 6 periodically. In one embodiment, a cover can be added on top of the influence tank 6 to prevent water evaporation.

In one preferred embodiment, recover the activity of discharged microorganisms might be necessary before recycling them to have higher performance once a while. A suitable recovery process includes washing, cleaning, rich medium culturing, combine with new inoculum or other efficient recovery processes. The microorganisms are removed from the aqueous phase if necessary. Cells are washed to avoid toxicity. Cell separation, if desired, is accomplished by centrifugation and membranous ultrafiltration, or other techniques. Microorganisms are recycled and maintained at high cell concentrations, thus maximizing reaction rates.

### Cheese Cage Electricity Collector and Connection

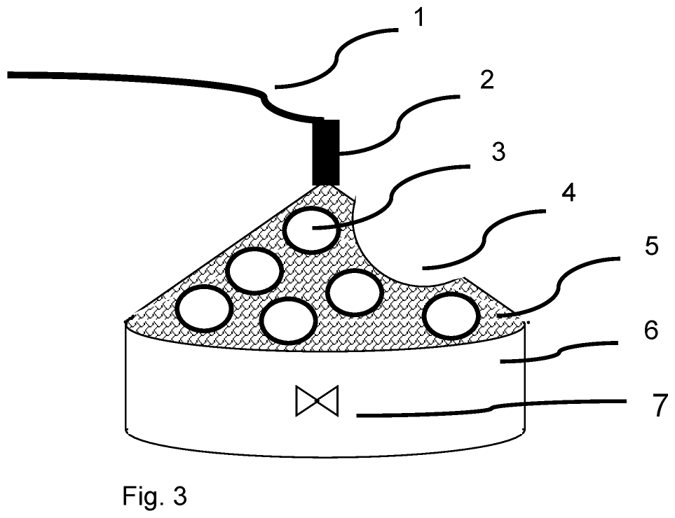

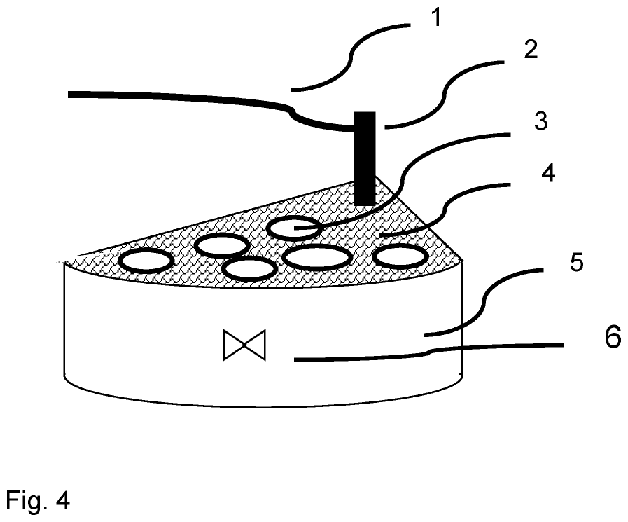

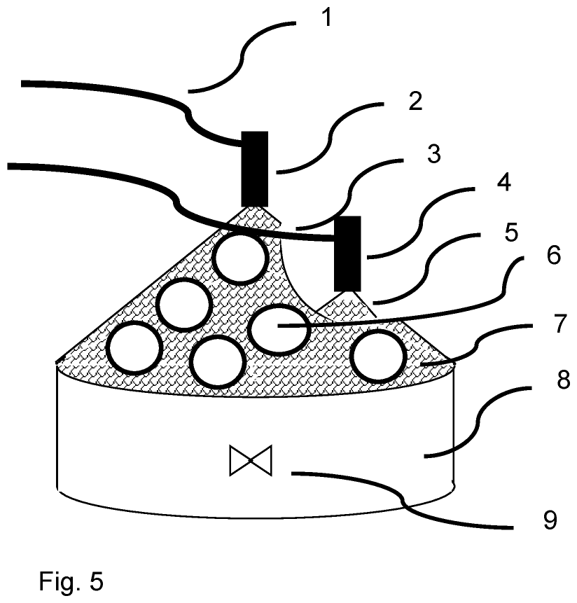

The biological battery manufacturing metal oxide system, wherein the anode and the cathode are connected to the external circuit through electricity collector and connectors, wherein is a design of cheese cage electricity collector (cheese cage collector) (FIG. 3) to enhance the efficiency of the current collection and transport in the biobattery reactor. It is in conical shape and is made of conductive and anticorrosive material, for example, graphite, graphene, metal, or mixture of metal or other compounds etc. As shown in FIG. 3, the structure is with a few holes 6 on the conical structure to allow path through pipelines for the reactor settings of liquid and gas flows. There is a big hole 4 on top of the conical structure, to allow sticking out of the counter electrode from inside of the structure, wherein the cone surface is solid, or net patterned 5 for better current flow. The structure contacts the electrode through the bottom cylinder 6, and an adjusting knob 7 to tighten the contact between the collector and the electrode to ensure a smooth current flow to the electrodes without a gap. A contacting tip 2 is on the top central of the conical structure for wiring 1 to the external circuit. FIG. 4 is the cheese cage collector of the counter electrode, wherein it has the same configuration and the position as of the counter electrode cheese cage collector to ensure they match each other when installed, except that the top tip 2 is away from the central location to avoid contact with the other electrode and short circuit when two cheese cage collectors installed on both electrodes in the system as shown in FIG. 5. Wherein the two cages are installed and 2 is the tip of one cage to an electrode and 4 and 5 are the tip and top of the cage to the counter electrode. They are separated. In an example of the biobattery reactor system FIG. 7. Both anode cheese cage and cathode cheese cage are installed, 1 is the wire connect to the cathode cheese cage tip 2, 3 is the wire connect to anode cheese cage tip 4.

### Nano Metal Oxide Structure Formation

An industrial method is provided for the synthesis of metal oxide for example Zinc oxide, ZnO, nanoparticles by a simple and economical wet chemical precipitation reaction between metal chloride, Zinc Chloride (ZnCl₂) and Sodium Hydroxide (NaOH) at low temperatures less than 80° C. (Radzimska 2014). Synthesis of ZnO Nanoparticles. Zinc oxide nanoparticles were synthesized by a modified wet chemical method involving the precipitation of zinc hydroxide, Zn(OH)₂, from the reaction of 0.1 mol zinc nitrate and 0.2 mol sodium hydroxide solutions followed by overnight drying of the precipitates at 80° C. to form ZnO (Rajan 2018). Ever the electrophoretic deposition method is used for the preparation of both ZnO think films and micro pattens. By applying small DC voltages between a Cu electrode and a conductive Si substrate, submerged in a suspension of ZnO quantum dots. ZnO layers of a tuneable thickness ranging from a few monolayers to 200 nm (Lommens 2008).

However, utilizing the electrochemical reaction in battery to form metal hydroxide first then using low heat dry (around 80° C.) to form metal oxide is a different way of making the metal oxide. In our method, electrochemical battery system is used, to obtain high quality metal oxide product, pure metal is placed as anode electrode with air in cathode in the battery system, under the neutral condition, without involving other metal and harsh chemicals, the metal hydroxide is produced. The process is renewable and clean and generate electricity at the same time. Metal hydroxide is converted to metal oxide at low temperature and with nano treatment to form interesting nano metal oxide structures. The nano structures formed in the metal oxide material products is advanced in the product quality compared to traditional combustion manufacture process.

Biobattery reactor system to produce metal oxide has advantages compared to using battery system. Firstly, biobattery operate under pH neutral condition. Without harsh acid and base condition, it is safer for the environment and human. Secondly, biobattery using biological medium to catalyze the electrochemical reaction, the process is long lasting, renewable, and low cost. Thirdly, certain functional microorganisms can fix CO₂ and produce biofuel and other useful byproducts of multi-carbon compounds. Fourthly, most of the effluent are recyclable, saves resources, cost of post treatment and environmental burden. The metal oxide material is produced during the biobattery electrochemical reaction while power is generated. The method of using biobattery electrochemical reaction to form the metal oxide material has advantage of forming interesting nano metal oxide structures during treatment compared to traditional combustion manufacture process.

### Examples

### Biological Battery Reactor Manufacturing Zinc Oxide

The functional microorganisms. The invention can be described specifically and illustrated by presenting one or more preferred embodiments in the paragraphs that follow. At least one preferred embodiment of the present invention provides for incorporating into an electrode one or more strains of microorganisms that can enact cathode catalyzing performance. The microorganisms catalyze the oxygen redox reaction on cathode forming hydroxide group and react with cations from anode forming useful oxides material. In at least one embodiment of producing ZnO. Unlike the traditional manufacture processes, which are energy intensive and generates lots of CO₂, especially combustion method, the biobattery metal oxide manufacture process can generates electricity, decarbonate while producing useful end products clean instead of consume electricity and generating CO₂ during the traditional manufacture process.

In at least one embodiment, the microorganisms could be photosynthetic mixotrophic microorganisms. They can fix CO₂ and convert carbon dioxide into biofuel or useful multi-carbon compounds under light and aerobic conditions (Wan 2015).

In at least one other embodiment, a microorganism can utilize the electrons from the cathode electrode as energy source and carbon from environment as carbon source to catalyze the electrochemical reactions near or on the cathode and promote the energy generation from the battery. The microorganisms also catalyze the electrochemical reaction in the battery in producing ZnO compounds. (Zhang 2020, 2023)

In at least one other embodiment, a microorganism can utilize the electrons from the cathode electrode as well as the energy from photons as energy source and carbon from environment as carbon source to catalyze the electrochemical reactions in battery. (Zhang 2020) Therefore promote the energy generation as well as Zinc oxide productions in the system. The microorganisms can be utilized in clean industrial metal oxide production while fulfil decarbonate and electricity generation at the same time.

In at least one embodiment, the reaction medium can be configured to contain carbon dioxide as a source of carbon and to be oxidized; a source of electrical energy, a source of light, the source of electrical energy connected to the cathode contact terminal; and the biological medium adjacent to at least one surface of the cathode electrode by way of the working medium.

Depending on the specific microorganism(s) utilized, variables to be considered in forming products include, for example, among others, without limitation: nutrient constituents and concentrations, medium, temperature, gas flow rate, liquid flow rate, access of light, access of CO₂, access of air, reaction pH, agitation rate (if utilizing a Continuously Stirred Tank Reactor), hydraulic retention time, inoculums concentration, maximum substrate (introduced gas) concentrations to avoid inhibition, and maximum product concentrations to avoid inhibition. The invention also provides, in an alternative aspect, one or more pure cultures of the microorganisms can be describable.

### Biological Battery (Biobattery) Reactor Design for Manufacturing ZnO

To promote production rate of ZnO and power output, it is important to have a proper biobattery design to provide better conditions for the functional microorganisms to grow and catalyze the electrochemical reactions. In one embodiment, wherein a reaction vessel, an anode electrode and a cathode electrode are all in cylinder shape as shown in FIGS. 1a and 1b. Wherein the anode electrode 1 is the outside cylinder; and the cathode electrode 3 is the inside cylinder, wherein inside cathode circle. encloses a biological medium 5, wherein in the middle of the biological medium is a light source 4 in the case of the biological medium contains photosynthetic CO₂ fixing microorganisms. these microorganisms need light to generate biological energy for fixing CO₂ providing biochemical reactions and biomass growth. Certain microorganisms can use the electrons from electrode as energy source. The biobattery reactor contains an electrolyte 2. In one embodiment, the electrolyte can mix with the biological medium. In another embodiment the electrolyte can separate from the biological medium. The electrolyte is in between the anode and cathode cylinders and contacting the anode and cathode electrodes directly. The biological medium is inside the cathode cylinder if separate from the electrolyte. A supporting and secure structure is at outside the anode electrode cylinder to physically support the shape of the reactor.

The advantages of this design are firstly for photosynthetic microorganisms, it is easier to control and monitor the light source, keep it constant for a better and consistent CO₂ fixation as well as manufacture of metal oxide and energy generation. Secondly this design separates the microorganism from the electrolyte and Zn(OH)₂ precipitation formed in the reactor. This design makes it better to control the electrolyte mass flow and the biological medium mass flow, recycle and keep a long-term better performance for both. And the design makes it easier to clean and wash the Zn(OH)₂ and finally product of ZnO product for a better quality. The disadvantage of this design is firstly, it limits the surface area of cathode and potentially limits the power output and energy density of the system. Secondly, the use of anode Zn might not be as efficient and cost effective due to it may need to customize the material.

In one other embodiment, wherein a reaction vessel as shown in FIG. 2, an anode and a cathode electrode are all in cylinder shape. The anode electrode 4 is the inside cylinder. The cathode electrode 3 is the outside cylinder. The outside cylinder 1 encloses the cathode electrode from outside with the biological medium 2 surrounding the cathode electrode and within the outside cylinder. The outside cylinder can be transparent in favored of photosynthetic CO₂ fixing microorganisms. The electrolyte 5 can mix with biological medium in one embodiment and separate from the biological medium in one other embodiment. The electrolyte is in between the anode and cathode and contacting the anode and cathode electrodes directly.

The advantage of this design is firstly it provides larger surface area for cathode have better power output and energy density. Secondly, it uses the anode Zn material more efficiently and cost effectively. The disadvantage is firstly, it is not easy to control and monitor the light source, therefore may impact the performance of the functional photosynthetic microorganisms.

### Biobattery Reactor System Design in Manufacturing ZnO

A biobattery reactor system in manufacturing ZnO product has mixed function of ZnO production, energy generation and CO₂ fixation. In one embodiment, the overall system combing the electrochemical battery technology and microbial syntrophic growth technology, displayed in an embodiment of biobattery system design as shown in FIG. 6. Where the functional microorganisms are grow in a reaction vessel with a Zn anode electrode 20 and a cathode electrode (graphite) 14 disposed therein, the Zn electrode having at least one surface and an anode electrical contact terminal, the cathode electrode (graphite) having at least one surface and a cathode electrical contact terminal, the cathode electrode having a biological medium 19, which contains biologically active material, adjacent to at least one modified surface of the cathode electrode and in electrical communication therewith, the reaction vessel configured to contain a working medium, electrolyte 17, for anode and cathode having mobile ions and in contact with the cathode electrode (graphite) and the anode electrode (Zn). the biological medium 19 contains functional microorganisms. The biological medium is configured to contain dissolved carbon dioxide as a source of carbon for cathode with CO₂ purging through pipeline 4. to near the cathode side, such that the gas mixes dissolve into the medium, and maintaining pH around neutral and maintaining other conditions appropriate for permitting the bacterium to catalyze the oxidation reaction with oxygen as electron acceptor and CO₂ as the carbon source. Also, there is a continuous access or supply of air or CO₂ into the biological medium in biological medium chamber 6 in influence tank as in FIG. 6.

### Medium Flow

A batch or a flow of medium into the reactor from an influence tank 8. The electrolyte from electrolyte chamber 7 in influence tank flow into electrolyte chamber 18 in biobattery reactor and flow out of the reactor through line 12. The biological medium in influence tank biological medium chamber 6 flow into the biological medium chamber 19 in biobattery reactor through pipeline 10 and flow out of the reactor through a pipeline 15 to recycle or move to collection tank 17.

In one embodiment, the working medium and the biological medium is one solution. It contains electrolyte, mobile ions, and cathode electrode active biological material (U.S. Utility patent Ser. No. 10/658,692, Awarded May 2020) In this embodiment the influence working medium flow into the biobattery reactor, wherein electrochemical reactions happened. And after the Zn(OH)₂ precipitation formed in certain volume, the working medium including the electrolyte, the Zn(OH)₂ precipitation and biological medium will be discharged as effluent to a wash and clean tank, wherein the Zn(OH)₂ precipitation will be washed to remove other chemicals and particles. Next step is to go through sieve, wherein the Zn(OH)₂ precipitation will be retained, and the small compounds and particles such as microorganisms will be sifted out. After filtration, the product will be air dry or dry through low heat around 80° C. to remove water molecules and form ZnO final product, which will be packed.

The wash out solution from the above process can be recycled after recovery via filtration through 0.2 μm filter to remove small particles including microorganisms if necessary, adding ions and adjust pH then add back to electrolyte in the biobattery or properly dispose.

In one other embodiment, the working medium electrolyte and the biological medium are separate solutions and are enclosed in separate compartment in the biobattery system. In FIGS. 1a and 1b, the electrolyte 2 are separated from biological medium 5.

In FIG. 6, a batch, or a flow of medium from an influence tank 8 into the biobattery reactor 21. The electrolyte from electrolyte chamber 7 in influence tank flow into electrolyte chamber 18 in biobattery reactor through pipeline 11 and flow out of the reactor through line 12. The biological medium in influence tank biological medium chamber 6 flow into the biological medium chamber 19 in biobattery reactor through pipeline 10 and flow out of the reactor through a pipeline 15 to recycle or move to collection tank 17.

The biological medium system can be a flow through system, as in FIGS. 6 and 7, in which a medium with biological active material flow from the biological medium compartment 6 in the influence tank through connecting pipeline 10 with a control valve 13, and into the biobattery reaction vessel 21, wherein biological medium catalyzing electrochemical reactions to form metal hydroxide, electricity and for certain microorganisms, biofuel or multi-carbon compounds as well. The effluent biological medium flow into a biological medium recycling pipelines 15, wherein the pipeline is in transparent or semi-transparent material to allow photosynthetic CO₂ fixing microorganisms to grow and keep alive during transportation. The effluent biological medium can be recycled back to the biological medium enclosed in cathode electrode in biobattery reactor or discharged to a collection tank 17 through a control valve 13 to extract useful compounds. The biobattery reactor can be single stand or stack up or parallel with multiple reactors sharing the same influence tank and effluent collection tank. The medium can be in the form of liquid or gel.

In regarding to the flow of the electrolyte. In the embodiment that the electrolyte is separated from the biological medium, as shown in FIGS. 1a and 1b, the electrolyte 7 in the influence tank 8 is separated from the biological medium 6. The electrolyte flow into the electrolyte compartment 18 in the biobattery reactor 21 through pipeline 11 and controlled by valve 13. In the biobattery reactor, the electrolyte is a solution in contact with the anode and cathode directly. It contains chemical compounds, mostly are ions and have major electrochemical reactions happened in it. After the metal is oxidized on anode and the oxygen is reduced on cathode, anions and cations migrate inside the electrolyte towards each other and form metal hydroxide precipitations mostly under the controlled conditions, such as pH, temperature, compounds in the electrolyte. At the same time, electrons are moved from cathode to anode via an external circuit through resistances or stored in another energy storage devices. The volume of anode will reduce over the time of the electrochemical reaction and the precipitation will be accumulated in the biobattery over the time. When certain volume of Zn(OH)₂ precipitation is formed, the control valve 13 will be opened, the Zn(OH)₂ precipitation, and the electrolyte will be moved out of the biobattery reactor into a wash and clean tank as shown in FIG. 8, wherein the Zn(OH)₂ precipitation will be washed and cleaned to remove other chemicals and particles. Next step is the filtration, wherein the Zn(OH)₂ precipitation will be retained, and the small compounds and particles will be filtered through. After filtration, the product will be air dry or dry through low heat to remove water molecules to final product ZnO, which will be packed. The wash out solution from the above process can be recycled after recovery via filtration through 0.2 μm filter, adding ions and adjust pH then add to electrolyte in the biobattery or properly dispose.

### CO₂ Fixation

The present invention provides, in at least one embodiment, a process for producing useful multi-carbon compounds, comprising biofuel: providing a continuous flow of air and/or a gas comprising carbon dioxide into a bioreactor, said bioreactor containing anode and cathode electrode, an aqueous nutrient biological medium and the microorganisms on the cathode side; directing a flow of the aqueous biological medium into the cathode side; and converting the gas in the medium by the microorganism at a pH around neutral and under conditions that permit converting the CO₂ to biofuel and useful multi-carbon compounds. Referring to one embodiment of a biological battery producing metal oxide material manufacture design of FIG. 6 and FIG. 7, CO₂ in pipe 4 is continuously purged into the biological medium 6 in the influence tank 8 as well as into the biological medium 19 of the biobattery reactor. The CO₂ gas is flow from the CO₂ tank 3 and delivered through pipe 4. The gas flow was controlled by regulator 2 correspondingly and through bubble counter 1 to monitor and deliver the CO₂ gas to the influence tank 8 and biobattery reactor 12 consistently and precisely. CO₂ flow rate is crucial for the biomass growth, therefore impact the production of the metal oxide compounds, the electricity and biofuel or multi-carbon compounds. This is because insufficient CO₂ supply may cause the inhibition of growth of the microorganisms during photosynthetic mixotrophic growth without sufficient carbon source. But over supply of CO₂ may cause pH change of the biological medium as well as the physiological growth condition of the functional group.

In one preferred embodiment the biofuel or multi-carbon compounds can be harvested from discharge the aqueous biological medium from the biobattery reactor. Referring to one embodiment of a biological battery producing ZnO material manufacture design of FIG. 6 and FIG. 7, the effluent of the biological medium from the biological medium tank 19 in the biobattery reactor 21 is recycled back to the biological medium 6 in the influence tank 8, through pipeline 15. A circulator pump 16 is installed on pipeline 15 to assist the circulation. The biofuel or multi-carbon products as well as biomass can also be discharged to the biofuel collection tank 17 through a control valve 13. In one preferred embodiment, the pipelines 15 and 10 are transparent. Influence biological medium can be directly added into influence tank 6 periodically. In one embodiment, a cover can be added on top of the influence tank 6 to prevent water evaporation.

In one preferred embodiment, recover the activity of discharged microorganisms might be necessary before recycling them to have higher performance once a while. A suitable recovery process includes washing, cleaning, rich medium culturing, combine with new inoculum or other efficient recovery processes. The microorganisms are removed from the aqueous phase if necessary. Cells are washed to avoid toxicity. Cell separation, if desired, is accomplished by centrifugation and membranous ultrafiltration, or other techniques. Microorganisms are recycled and maintained at high cell concentrations, thus maximizing reaction rates.

### Cheese Cage Electricity Collector and Connection

The biological battery manufacturing ZnO system, wherein the anode Zn and the cathode of graphite are connected to the external circuit through electricity collector and connectors, wherein is a design of cheese cage electricity collector (cheese cage collector) (FIG. 3 or FIG. 4) to enhance the efficiency of the current collection and transport in the biobattery reactor. It is in conical shape and is made of conductive and anticorrosive material. for example, graphite, graphene, metal, or mixture of metal or other compounds etc. As shown in FIG. 3, is the cheese cage collector of the cathode graphite electrode. The structure is with a few holes 6 on the conical structure to allow path through pipelines for the reactor settings of liquid and gas flows. There is a big hole 4 on top of the conical structure, to allow sticking out of the anode Zn electrode from inside of the structure, wherein the cone surface is solid, or net patterned 5 for better current flow. The structure contacts the electrode through the bottom cylinder 6, and an adjusting knob 7 to tighten the contact between the collector and the electrode to ensure a smooth current flow to the electrodes without a gap. A contacting tip 2 is on the top central of the conical structure for wiring 1 to the external circuit. FIG. 4 is the cheese cage collector of the anode Zn, wherein it has the same configuration and the position as of the cathode cheese cage collector to ensure they match each other when installed, except that the tip 2 is away from the central location to avoid contact with the other electrode and short circuit when two cheese cage collectors installed on both electrodes in the system as shown in FIG. 5. Wherein the two cages are installed and 2 is the tip of the cathode cage. 1 is the wire connect to the tip of the cathode cage. 4 and 5 are the tip and top of the anode cage. 3 is the wire connect to the anode cage tip 4. Anode cage and the cathode cage are separated and not touched. In an example of the biobattery reactor system FIG. 7. Both anode cheese cage and cathode cheese cage are installed, 1 is the wire connect to the cathode cheese cage tip 2, 3 is the wire connect to anode cheese cage tip 4.

### Nano Metal Oxide Structure Formation

An industrial method is provided for the synthesis of metal oxide for example Zinc oxide, ZnO, nanoparticles by a simple and economical wet chemical precipitation reaction between metal chloride, Zinc Chloride (ZnCl₂) and Sodium Hydroxide (NaOH) at low temperatures less than 80° C. (Radzimska 2014). Synthesis of ZnO Nanoparticles. Zinc oxide nanoparticles were synthesized by a modified wet chemical method involving the precipitation of zinc hydroxide, Zn(OH)₂, from the reaction of 0.1 mol zinc nitrate and 0.2 mol sodium hydroxide solutions followed by overnight drying of the precipitates at 80° C. to form ZnO (Rajan 2018). Ever the electrophoretic deposition method is used for the preparation of both ZnO think films and micropattens. By applying small DC voltages between a Cu electrode and a conductive Si substrate, submerged in a suspension of ZnO quantum dots. ZnO layers of a tuneable thickness ranging from a few monolayers to 200 nm (Lommens 2008).

However, utilizing the electrochemical reaction in battery to form Zn(OH)₂ first then using low heat dry (around 80° C.) to form ZnO is a different way of making the ZnO. In our method, electrochemical battery system is used, to obtain high quality ZnO product, pure metal Zn is placed as anode electrode with air in cathode graphite in the battery system, under the neutral condition, without involving other metal and harsh chemicals, the Zn(OH)₂ precipitation is produced. The process is renewable, clean and generate electricity at the same time. Zn(OH)₂ is converted to ZnO at low temperature and with treatment to form interesting nano ZnO structures. The nano structures formed in the ZnO material is advanced in the product quality compared to traditional combustion manufacture process.

Biobattery reactor system to produce ZnO has advantages compared to using battery system. Firstly, biobattery operate under PH neutral condition. Without harsh acid and base condition, it is safer for the environment and human. Secondly, biobattery using biological medium to catalyze the electrochemical reaction, the process is long lasting, renewable, and low cost. Thirdly, certain functional microorganisms can fix CO₂ and produce biofuel and other useful byproducts of multi-carbon compounds. Fourthly, most of the effluent are recyclable, saves resources, cost of post treatment and environmental burden. The metal oxide material is produced during the biobattery electrochemical reaction while power is generated. The method of using biobattery electrochemical reaction to form the metal oxide material has advantage of forming interesting nano metal oxide structures during treatment compared to traditional combustion manufacture process.

### Other Applications

In addition, the present invention can be used as cathode configuration of many devices and applications. Modifications to the conditions can result in the generation of higher current density. The Microbial Fuel Cell can comprise a reaction vessel having an anode electrode/stack of anode electrodes and a cathode electrode/stack of cathode electrodes disposed therein. At least one embodiment of the invention provides further for no ion exchange membrane or other separators to be placed in between the anode and cathode electrode, the cathode electrode having biological medium adjacent to at least one surface of the cathode electrode and in electrical communication therewith. The reaction vessel configured to contain an electrolyte in contact with anode and one side of cathode therein; and a biological medium contains active biological material in contact with the cathode electrode. Referring to one embodiment of a biological battery producing metal oxide material manufacture design of FIG. 1a, electrolyte solution 2 is contained in between anode 1 and cathode 3. The biological medium 5 was enclosed inside of a cylinder cathode 3. A light source 4 was placed in the middle of the biological medium 5 to uniformly deliver the light in the biological medium. Referring to FIG. 1b is the side view of the same configuration.

### REFERENCES

Fokotsa V. M., Lehlohonolo F. K., Birhanu F. D., Hendrik C. S. (2015). “Phase formation of hexagonal wurtzite ZnO through decomposition of Zn(OH)₂ at various growth temperatures using CBD method.” Optical Materials 46 (2015) 292-298
Kolodziejczak-Radzimska A., Jesionowski, T., Krysztafkiewicz, A., “Obtaining Zinc Oxide From Aqueous solutions of KOH AND Zn(CH₃COO)₂.” Physicochem. Probl. Miner. Process. 2010 44 (2010) 93-102
Kolodziejczak-Radzimska A., Jesionowski, T. (2014). “Zinc Oxide—From Synthesis to Application: A Review.” Materials 2014 (7): 2833-2881.
Lommens, P., Thourhout, D., Smet, D., Poelman, D and Hens., Z. (2008). “Electrophoretic deposition of ZnO nanoparticles, from micropatterns to substrate coverage.” Nanotechnology. 2008 Jun. 18;19 (24): 245301
Rajan, A., Singh A. (2018). Synthesis of Zinc Oxide Nanoparticles by Wet Chemical Method and Study of Enhancement of The Antimicrobial Activity of Various Antibiotics by Zinc Oxide Nanoparticles.” International Journal of Engineering Research & Technology (IJERT). 2018 ISSN: 2278-0181
Semenova, A. A., Lashkova, N. A., Maximov, A. I., and Moshnikov, V. A. (2017). Formation of one-dimensional ZnO structures on flexible substrates, Journal of Physics: Conf. Series 816. 2017. DOI 10.1088/1742-6596/816/1/012031
Scheinost, A. C. (2005). “Metal Oxides.” Encyclopedia of Soils in the Environment.
Wan, N., Abernathy, M., Tang, J., Tang, Y., You, L. (2015). “Cyanobacterial photo-driven mixotrophic metabolism and its advantages for biosynthesis Front. Chem. Sci. Eng. 2015. DOI 10.1007/s11705-015-1521-7
Wang, M., Zhou, Y., Zhang, Y., Hahn, S. H., and Kim, E. J., (2011). “From Zn(OH)₂ to ZnO: a study on the mechanism of phase transformation.” CrystEngComm, 2011, 13, 6024. DOI: 10.1039/c1ce05502j
Zhang, P., Girguis, P. (2012). Development of advanced biocathode for enhancement of current density of Microbial Fuel Cells. NSF award final report Award #1214797.
Zhang, P. (2020). Biological Battery and Biological Cathode. Utility patent U.S. Ser. No. 10/658,692
Zhang, P. (2023). Biological Battery and Biological Cathode. Utility patent U.S. Ser. No. 11/575,171

What is claimed is:

1. A biological battery reactor system is in manufacturing of metal oxide, generating electricity and other compounds, wherein the system can be batch, semi-batch or continuous flow system and including:
an influence vessel or tank, configured to retain therein influence medium, and including:
an electrolyte, and
a biological medium, and
a biological battery reactor, configured to have metal air redox reaction to form metal oxide, generate electricity and other compounds, and including:
a cathode electrode, and
an anode electrode, and
an electrolyte, and
a biological medium, and
electricity collectors and connectors to the external circuit, and
energy transfer or storage devices or both, for example resistors or capacitor, and
pipelines connect the influence medium in the influence vessel and the medium in the biological battery reactor, and
a discharge or recycle of effluent products for different post treatment, and
a gas supply when it is needed to provide constant, individually controlled, or precise amount of CO₂ or other gases for utilizing by microorganisms or the system.

2. The biological battery manufacturing metal oxide system of claim 1, wherein the influence vessel or tank configured to retain therein influence medium, and including:
a medium including an electrolyte and a biological medium in the same compartment vessel, or
a biological medium in the one compartment of the vessel, and an electrolyte in another compartment of the influence vessel, wherein the biological medium and electrolyte are well separated.

3. The biological battery manufacturing metal oxide system of claim 1, wherein a biological battery reactor is configured to contain medium, wherein:
a mixture of an electrolyte and a biological medium is in the medium, wherein
it is in between the anode and the cathode electrodes, and
contacts the anode electrode and the cathode electrode; or
a separate of an electrolyte and a biological medium is configured, wherein
an electrolyte is in between the anode and the cathode electrodes. Wherein it contacts the anode electrode and the cathode electrode, and
a biological medium, contacts the cathode electrode only to catalyze battery reaction, and for CO₂ fixing microorganisms to form interesting biological synthesized multi-carbon compounds.

4. The biological battery manufacturing metal oxide system of claim 1, wherein pipelines connect the influence medium in the influence tank and the medium in the biological battery reactor, and including:
the mixture of an electrolyte, and a biological medium in the influence tank and in the biological battery reactor with valve control; or
the separate of an electrolyte in the influence tank and the electrolyte in the biobattery reactor with valve control; and the separate biological medium in the influence tank and the biological medium in the biobattery reactor with valve control.

5. The biological battery manufacturing metal oxide system of claim 1, wherein a discharge of effluent products, and including:
products of mixed metal hydroxide, metal oxide and multi-carbon compounds from biobattery, wherein
precipitation be filtered, washed, dried and treated to become the final product, and
multi-carbon compounds be filtered, extract to the end products or be filtered, and recycled back (after treatment if needed) to the biological battery reactor; or
a separate discharge of electrolyte with metal oxide and biological medium from the biological battery reactor, wherein
the electrolyte with precipitation is discharged alone into a tank, wherein the precipitation is filtered, washed, dried, treated to become final metal oxide product. Or the rest be recycled, and
the biological medium be discharged alone in multiple situations, wherein
completely to a collection tank of bio-synthesized multi-carbon compounds for further treatment and use; or
partially to the collection tank to extract the bio-synthesized multi-carbon compounds for further treatment and partially be recycled back to the biological medium (after treatment if needed) in the biological battery, wherein through transparent or opaque pipelines in case of growing certain photosynthetic CO₂ fixing microorganisms.

6. The biological battery manufacturing metal oxide system of claim 1, wherein a gas supply if needed to provide constant, individually controlled, or precise amount of CO₂ or other gases for utilizing by microorganisms or the system, a gas pipelines with regulator and bubble counting or flow control meters to wherein the places in the system, including:
for the system has electrolyte mixed with biological medium, any gas can be delivered to the medium chamber in influence tank and the medium chamber in biological battery reactor, wherein the electrolyte and the biological medium are mixed; or
for the system with electrolyte separately contained from biological medium, there are several situations, including:
if the electrolyte and the biological medium are separately contained, wherein
deliver CO₂ to the biological medium in the biological medium compartment in the influence tank and to the biological medium in biological medium compartment in biological battery reactor, and
deliver air to electrolyte compartment in influence tank or in electrolyte near cathode in biological battery reactor; or
if the electrolyte and the biological medium are mixed contained, wherein
deliver CO₂ to the biological medium in the biological medium compartment in the influence tank and to the biological medium in biological medium compartment in biological battery reactor; or
deliver air to places in the electrolyte close to cathode in the system; or
deliver CO₂ to the biological medium in the biological medium compartment in the influence tank and to the biological medium in biological medium compartment in biological battery reactor, and deliver air to places in the electrolyte close to cathode in the system.

7. The biological battery manufacturing metal oxide system of claim 1, wherein the biological medium in influence tank and biological battery is including:
one or a mix of multiple functional microorganisms; or
one or a mix of multiple growth agents; or
one or a mix of multiple biological agents, such as enzymes or biochemicals; or
one or a mix of microbial extracellular compounds; or
one or a mix of organic compounds; or
one or a mix of inorganic compounds; or
one or a mix of physical particles; or
a mix of any of the above.

8. The functional microorganisms of claim 7, including:
one or a mix of pure culture of autotrophic microorganisms; or
one or a mix of heterotrophic microorganisms; or
one or a mix of mixotrophic microorganisms; or
one or a mix of phototrophic microorganisms; or
one or a mix of photosynthetic mixotrophic CO₂ fixing microorganisms; or
a mix of any microorganisms of the above.

9. The biological battery manufacturing metal oxide system of claim 1, wherein one design of biological battery reactor is a biological battery reactor vessel, an anode electrode and a cathode electrode are all in cylinder shape, and including:
the anode electrode is the outside cylinder, and
the cathode electrode is the inside cylinder, wherein inside cathode circle, encloses:
a biological medium, wherein in the middle of the biological medium is including:
a light source in case of the biological medium contains functional microorganisms, and
a medium that is in between the anode and cathode cylinders and contacting the anode and cathode electrodes directly, and
a supporting and secure structure at outside the anode electrode cylinder.

10. The biological battery manufacturing metal oxide system of claim 1, wherein one other design of biobattery reactor is a biobattery reactor vessel, an anode and a cathode electrode are all in cylinder shape. and including:
the anode electrode is the inside cylinder; and a cathode electrode is the outside cylinder, and
an outside cylinder encloses the cathode electrode from outside with the biological medium surrounding the cathode electrode and within the outside cylinder, wherein is transparent in case of needed by the system with functional microorganisms, and
a medium that is in between the anode and cathode and contacting the anode and cathode electrodes directly.

11. The biological battery manufacturing metal oxide system of claim 1, wherein the anode and the cathode are connected to the external circuit through electricity collector and connectors, wherein is a design of cheese cage electricity collector (FIG. 3, FIG. 4, and FIG. 5) to enhance the efficiency of the current collection and transport and with designs, including:
in conical shape, and
made of conductive and anticorrosive material, and
with a few holes on the cone to allow path through pipelines for reactor setting, liquid and gas flow, and
a full circular, contacts the electrodes without a gap, and
a contact tip structure on the top of the conical structure for wiring to the external circuit, and
solid or net pattern of the conical top surface, and
Solid surface and conductive material of the conical side structure.

12. A method of using battery electrochemical reactions to produce metal oxide material with nano structures can be through batch or continuous battery systems and including:
a metal air battery set up, including:
a metal anode electrode as electron donor, and
a cathode using oxygen or air as electron acceptor, and
an electrolyte solution that contacts anode and cathode directly, and
an external circuit connect the anode and cathode through wire, and
energy transfer or storage devices or both, for example resistors or capacitor, and
filter, wash, and dry the precipitation from the metal air battery, and
treatment to form desired structured metal oxide compounds.

13. The biological battery manufacturing metal oxide system of claim 1, wherein the discharge of biological battery reactor of metal hydroxide is treated to form metal oxide material with nano structures through dissolution-reprecipitation, in situ crystallization or solid-solid phase transformation to form metal oxide.

14. A biological battery reactor system is in manufacturing of Zinc oxide, generating electricity and other compounds, wherein the system can be batch, semi-batch or continuous flow system and including:
an influence vessel or tank, configured to retain therein influence medium, and including:
an electrolyte, and
a biological medium, and
a biological battery reactor, configured to have metal air redox reaction to form metal oxide, generate electricity and other compounds, and including:
a cathode electrode, and
an anode electrode, and
an electrolyte, and
a biological medium, and
electricity collectors and connectors to the external circuit, and
pipelines connect the influence medium in the influence vessel and the medium in the biological battery reactor, and
a discharge or recycle of effluent products for different post treatment, and
a gas supply when it is needed to provide constant, individually controlled, or precise amount of CO₂ or other gases for utilizing by microorganisms or the system.

15. The biological battery manufacturing Zinc oxide system of claim 14, wherein a discharge of effluent products, and including:
products of mixed Zinc hydroxide, Zinc oxide and multi-carbon compounds from biobattery, wherein
precipitation be filtered, washed, dried and treated to become the final product, and
multi-carbon compounds be filtered, extract to the end products or be filtered, and recycled back (after treatment if needed) to the biological battery reactor; or
a separate discharge of electrolyte with metal oxide and biological medium from the biological battery reactor, wherein
the electrolyte with precipitation is discharged alone into a tank, wherein the precipitation is filtered, washed, dried, treated to become final metal oxide product. Or the rest be recycled, and
the biological medium be discharged alone in multiple situations, wherein
completely to a collection tank of bio-synthesized multi-carbon compounds for further treatment and use; or
partially to the collection tank to extract the bio-synthesized multi-carbon compounds for further treatment and partially be recycled back to the biological medium (after treatment if needed) in the biological battery, wherein through transparent or opaque pipelines in case of growing certain photosynthetic CO₂ fixing microorganisms.

16. The biological battery manufacturing Zinc oxide system of claim 14, wherein the discharge of biological battery reactor of Zinc hydroxide is treated to form zinc oxide material with nano structures through dissolution-reprecipitation, in situ crystallization or solid-solid phase transformation, or mix of any of them.

17. The biological battery manufacturing Zinc oxide system of claim 14, wherein a biological battery reactor is configured to contain medium, wherein:
a mixture of an electrolyte and a biological medium is in the medium, wherein
it is in between the anode and the cathode electrodes, and
contacts the anode electrode and the cathode electrode; or
a separate of an electrolyte and a biological medium is configured, wherein
an electrolyte is in between the anode and the cathode electrodes. Wherein it contacts the anode electrode and the cathode electrode, and
a biological medium, contacts the cathode electrode only to catalyze battery reaction, and for CO₂ fixing microorganisms to form interesting biological synthesized multi-carbon compounds.

18. The biological battery manufacturing Zinc oxide system of claim 14, wherein pipelines connect the influence medium in the influence tank and the medium in the biological battery reactor, and including:
the mixture of an electrolyte, and a biological medium in the influence tank and in the biological battery reactor with valve control; or
the separate of an electrolyte in the influence tank and the electrolyte in the biobattery reactor with valve control; and the separate biological medium in the influence tank and the biological medium in the biobattery reactor with valve control.

19. The biological battery manufacturing Zinc oxide system of claim 14, wherein a gas supply if needed to provide constant, individually controlled, or precise amount of CO₂ or other gases for utilizing by microorganisms or the system, a gas pipelines with regulator and bubble counting or flow control meters to wherein the places in the system, including:
for the system has electrolyte mixed with biological medium, any gas can be delivered to the medium chamber in influence tank and the medium chamber in biological battery reactor, wherein the electrolyte and the biological medium are mixed; or
for the system with electrolyte separately contained from biological medium, there are several situations, including:
if the electrolyte and the biological medium are separately contained, wherein
deliver CO₂ to the biological medium in the biological medium compartment in the influence tank and to the biological medium in biological medium compartment in biological battery reactor, and
deliver air to electrolyte compartment in influence tank or in electrolyte near cathode in biological battery reactor; or
if the electrolyte and the biological medium are mixed contained, wherein
deliver CO₂ to the biological medium in the biological medium compartment in the influence tank and to the biological medium in biological medium compartment in biological battery reactor; or
deliver air to places in the electrolyte close to cathode in the system; or
deliver CO₂ to the biological medium in the biological medium compartment in the influence tank and to the biological medium in biological medium compartment in biological battery reactor, and deliver air to places in the electrolyte close to cathode in the system.

20. A method of using battery electrochemical reactions to produce metal oxide of claim 12, the metal oxide, including:
Zinc Oxide, and
Magnesium oxide, and
Aluminum oxide, and etc.
